(19) United States
(12) Patent Application Publication
(10) Pub. No.: US 2026/0174603 A1
(43) Pub. Date: Jun. 25, 2026

(54) Folding machines and methods for manufacturing pant products

(71) Applicant: CURT G. JOA, INC., Sheboygan Falls, WI (US)

(72) Inventors: Christopher A. SCHWARTZ, Sheboygan Falls, WI (US); Jeffrey W. FRITZ, Sheboygan Falls, WI (US); Darren R. HORNESS, Sheboygan Falls, WI (US); Sean P. FOLLEN, Sheboygan Falls, WI (US)

(21) Appl. No.: 19/124,549

(22) PCT Filed: Oct. 10, 2023
(86) PCT No.: PCT/US2023/034829

§ 371 Date: Apr. 25, 2025

Related U.S. Application Data
(60) Provisional application No. 63/420,000, filed on Oct. 27, 2022.

(51) Int. Cl. A61F 13/15; A61F 13/496

(52) U.S. Cl. CPC A61F 13/15747; A61F 13/496

(57) ABSTRACT

A pant folding machine is configured to fold an intermediate pant product having first and second faces and formed by continuous webs of first and second waistband materials and cores. The machine includes a frame; an inlet roller configured to receive the intermediate pant product in an unfolded state; a folding apparatus; and an outlet roller configured to receive the intermediate pant product in the folded state. The folding apparatus includes a center board and first and second side boards. The boards are arranged such that the center board engages the first face of the intermediate pant product and each of the first and second side boards engages the second face of the intermediate pant product and, as the intermediate pant product is drawn along the boards, the intermediate pant product is folded by the boards to transition from the unfolded state to the folded state.

### CROSS-REFERENCE TO RELATED APPLICATION

This application claims priority to U.S. Provisional Patent Application No. 63/420,000, filed Oct. 27, 2022.

### FIELD OF THE DISCLOSURE

This disclosure generally relates to systems, machines, and methods for forming or manufacturing disposable undergarments or diapers, particularly pant-type products.

### BACKGROUND OF THE DISCLOSURE

Generally, disposable undergarments such as pant-type diapers (generally, “pant products”) are made up of two nonwoven layers with embedded elastic material that form front and back waistbands and an absorbent core that is inserted onto or within the waistbands to be positioned proximate to the body of a user. Such pant products are generally manufactured in an assembly or processing line of stations that perform various functions. For example, the cores may be layered and sectioned, and the waistbands may be formed from continuous webs of material onto which the cores are positioned. Subsequent processing steps may include cuff formation, bonding to create seams, cutting the continuous web of material into individual products, and/or packaging. Manufacturers of the pant products attempt to maintain or improve the speeds and efficiencies of various aspects of the system and process, as well as to provide improved product features.

### BRIEF DESCRIPTION OF THE DRAWINGS

FIG. 1 is a schematic representation of a system for manufacturing pant products, including a folding machine according to an example embodiment;

FIG. 2A is a representative plan view of an intermediate pant product that may be processed by the system for manufacturing pant products of FIG. 1 according to an example embodiment;

FIG. 2B is a representative isometric view of a finished pant product, unfolded, that may be produced by the system for manufacturing pant products of FIG. 1 according to an example embodiment;

FIG. 3 is a first isometric view of a folding machine of the system for manufacturing pant products of FIG. 1 according to an example embodiment;

FIG. 4 is a further isometric view of the folding machine of FIG. 3 according to an example embodiment;

FIG. 5 is a side view of a folding machine of FIG. 3 according to an example embodiment;

FIG. 6 is an isolated side view of a folding board that may be incorporated into the folding machine of FIG. 3 according to an example embodiment;

FIG. 7 is a first isolated isometric view of rollers and a folding apparatus that may be incorporated into the folding machine of FIG. 3 according to an example embodiment;

FIG. 8 is a further isolated isometric view of the rollers and folding apparatus that may be incorporated into the folding machine of FIG. 3 according to an example embodiment;

FIG. 9 is an isolated isometric view of the folding apparatus that may be incorporated into the folding machine of FIG. 3 according to an example embodiment;

FIG. 10 is a representative cross-sectional view of an intermediate pant product passing through the folding apparatus that may be incorporated into the folding machine of FIG. 3 according to an example embodiment;

FIG. 11 is a simplified representation of the intermediate pant product depicting schematic fold lines generated by the folding apparatus that may be incorporated into the folding machine of FIG. 3 according to an example embodiment;

FIG. 12 is a simplified representation of a folded intermediate pant product produced by the folding apparatus that may be incorporated into the folding machine of FIG. 3 according to an example embodiment;

FIG. 13 is a representative isometric view of a finished pant product, unfolded, that may be produced by the system for manufacturing pant products of FIG. 1 according to another example embodiment;

FIG. 14 is a first isolated isometric view of rollers and a folding apparatus that may be incorporated into a folding machine according to a further example embodiment;

FIG. 15 is a further isolated isometric view of the rollers and folding apparatus of FIG. 14 according to the further example embodiment;

FIG. 16 is an isolated isometric view of the folding apparatus of FIGS. 14 and 15 according to the further example embodiment;

FIG. 17 is a representative cross-sectional view of an intermediate pant product passing through the folding apparatus of FIG. 16 according to the further example embodiment;

FIG. 18 is a simplified representation of the intermediate pant product depicting schematic fold lines generated by the folding apparatus of FIG. 16 according to the further example embodiment;

FIG. 19 is a simplified representation of a folded intermediate pant product produced by the folding apparatus of FIG. 16 according to the further example embodiment; and

FIG. 20 is a side view of a folding board and band configuration that may be incorporated into the folding machine of FIG. 1 according to a further example embodiment.

Like reference symbols in the various drawings indicate like elements.

### DETAILED DESCRIPTION

The following describes one or more example embodiments of the disclosed folding machines and methods for manufacturing disposable pant products, as shown in the accompanying figures of the drawings described briefly above. Various modifications to the example embodiments may be contemplated by one of skill in the art.

In the embodiments discussed herein, representations of directions or orientations (e.g., up, down, front, forward, side, upper, lower, distal, rear, inner, and outer) may be used for explaining the structure and configurations of the various elements, but these representations should be considered as relative to one another in the particular examples and should not be considered as absolute limitations. Additionally, the discussion below may reference a material or product-based coordinate system with respect to continuous webs in the primary direction of movement, e.g., with a longitudinal orientation being parallel to the direction of movement at a respective location, a lateral orientation being “side-to-side”, perpendicular to the longitudinal orientation within the plane of the product, and a transverse orientation being perpendicular to the longitudinal and lateral orientations. In other instances, the discussion below may reference an absolute coordinate system relative to the particular orientation of the machine (e.g., x, y, and z).

As introduced above, disposable undergarments such as pant-type diapers (generally, “pant products”) are made up of two nonwoven layers with embedded elastic material that form front and back waistbands and an absorbent core that is inserted onto or attached to the waistbands to be positioned proximate to the body of a user. Such pant products are generally manufactured in an assembly or processing line of stations that perform various functions. For example, the cores may be layered and sectioned, and the waistbands may be formed from continuous webs of material onto which the cores are positioned. Subsequent processing steps may include cuff formation, bonding to create seams, cutting the continuous web of material into individual products, packaging, and/or other steps, particularly folding, as discussed in greater detail below.

In some previous examples, the pant products, while in the continuous web, are folded a single time in a lateral center position (e.g., along a longitudinal centerline) to result in an asymmetric intermediate pant product in which the core is roughly on one lateral side of a resulting longitudinal centerline and the two waistbands are roughly on the other lateral side prior to side seam bonding and separation. This configuration not only results in a relatively “wide” intermediate pant product, but it may result in a center of mass that is not along the subsequent or folded longitudinal centerline (and/or, generally, a poorly distributed mass), which may raise issues during such folding. Moreover, the asymmetric nature of this web with a single fold may present issues with handling the continuous web, particularly considering the desire to manufacture a large number of pant products in a short amount of time. Such issues with the asymmetrical web May necessitate slower speeds, and/or the asymmetrical web may require additional control mechanisms, such as control belts to hold the portions with the cores in place while being accelerated and decelerated through downstream processes.

Example embodiments described herein are directed to a folding machine and method in which the cores of an intermediate pant product are tucked up in between the waistbands of the continuous web materials, prior to forming the seams and separation into discrete products. In effect, the folding machine may use a set of three boards in which a center board forms a fold approximately along the longitudinal centerline of the core (and the overall, unfolded intermediate pant product) on a first face or surface of the intermediate pant product and a pair of side boards, each on a respective side of center board, engage the other face of the intermediate pant product to form side folds. This configuration results in a “W” or “M” shaped tri-fold in which the core is tucked in between the waistbands, thereby providing a laterally symmetrical folded intermediate pant product. The resulting, more evenly distributed mass provides a more stable intermediate pant product for further processing, including mitigating or eliminating the need for control mechanisms (e.g., control belts) and/or simplifies downstream processing, including subsequent cutting and transport steps. For example, the tri-fold configuration may reduce or eliminate the need for further folding of the discrete pant products. In general, the examples embodiments discussed herein may provide a more robust, stable, and faster manufacturing capability. Moreover, in some configurations, the folding machine 3 may enable and/or facilitate the placement of seams on the inner face of the pant 4 product, thereby providing an improved customer experience.

It is noted that the present folding techniques, machines, systems, apparatus, and methods are described herein with respect to products such as brief or pant-type diapers or undergarments for babies, infants, children, or adults. However, such examples may be applied to a wide variety of processes in which a continuous web of products are folded prior to further processing steps, such as bonding and cutting.

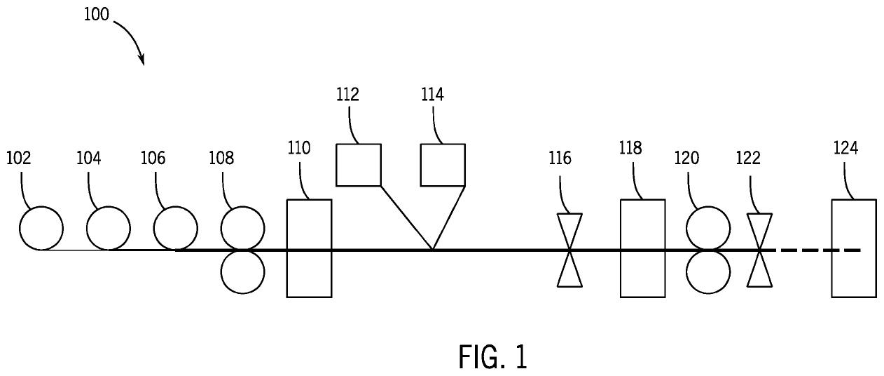

Reference is now made to FIG. 1, which is a schematic representation of a system or arrangement 100 for manufacturing or forming pant products with a number of stations, sections, machines, and/or units 102, 104, 106, 108, 110, 112, 114, 116, 118, 120, 122, 124. Initially, it is noted that the system 100 of FIG. 1 is merely an example, and the embodiments discussed below, particularly as it pertains to folding, are applicable to other or modified systems, machines, and/or processes. Generally, subject to the discussion below, the system 100 and the associated sections 102, 104, 106, 108, 110, 112, 114, 116, 118, 120, 122, 124 may have any suitable equipment for forming the pant products, including material sources, treatment equipment, belts, cutting implements, drums, conveyors, guides, manipulators, and the like. For example, one or more controllers may be provided to facilitate operation and/or automation of the manufacturing, assembly, treatment, folding, and/or packaging processes. One example of an overall configuration of the system 100 is discussed below prior to a more detailed description of the folding machine 118. In some instances, details about certain aspects of the overall configuration may be omitted for brevity or clarity.

Sections 102, 104, 106 represent core (or insert) forming and processing units that function to produce a two layer or “dual” primary structure with one or more additional layers or features, such as a topsheet (e.g., for skin contact), an acquisition layer (e.g., to facilitate wicking), a backsheet, a debulked structure, and/or embossed pattern. In other examples, the core may have only a single layer, in which case section 102 or 104 may be omitted, or may have more than two primary layers. Generally, the cores may be formed from one or more absorbent materials, such as pulp, paper, polymer, or tissue materials. Additionally, section 108 functions as a bonding station to form a cuff section attached to each core via ultrasonic or adhesive bonding means, and may optionally further function to transfer the cores as a continuous stream, which are later separated into discrete pieces for assembly.

Sections 110, 112, 114 represent joining or processing units in which front and rear waistbands are joined with the core. In particular, the waistbands may be formed and/or otherwise provided by sections 112, 114 in preparation for assembly with the cores. In general, two waistbands (e.g., corresponding to front and rear waistbands of the finished product) may be formed from any suitable type of material, such as a non-woven fabric material that may be ultrasonically or adhesively bonded to entrap contain strands, bands, or films/sheets of elastic material, thereby, in this example, resulting in two continuous webs of waistband material. Alternatively, the waistband material may be manufactured in an offline process and provided to the machine as a preformed, single or multi-layer elasticized composite material. Moreover, in some examples, the front and rear waistbands may be initially formed from the same material web and then cut to form the separate waistbands. Regardless, the continuous webs of front and rear waistband materials are transferred to downstream stations. In one example, section 110 may be considered one or more machines functioning to separate, rotate, and repitch the discrete cores and facilitate core placement, e.g., with an adhesive, to form a stream of intermediate pant product.

Further processing of the intermediate pant product occurs in sections 116, 118, 120. In particular, section 116, which may be omitted in some embodiments, represents a cutting unit in which portions of waistbands are cut to create curved leg openings. Section represents a folding machine (generally, folding machine 118) in which the continuous stream of intermediate pant product is folded in three places with an assembly or array of folding boards, as discussed in greater detail below. Section 120 represents one or more bonding units in which the folded intermediate pant products are ultrasonically bonded to form side seams. In alternate embodiments, side seams may be formed with a single bonding unit or additional bonding units may be provided. In yet other embodiments, side seams may be formed with adhesive.

Section 122 represents a cutting or knife unit in which the continuous web of intermediate pant product is separated into discrete pieces at the approximate centers of the side seams. Finally, in this example, section 124 represents a transfer unit in which the discrete pieces of intermediate pant product are picked up and turned into a packaging orientation, and may further represent a packaging folding unit in which the discrete intermediate pant products are folded for packaging.

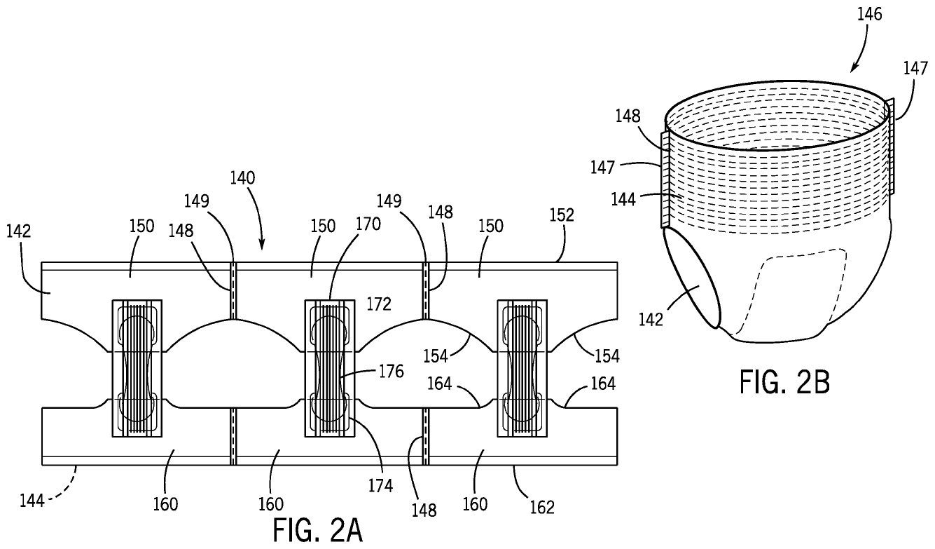

As such, the pant manufacturing system 100 operates to form finished pant products from the various continuous (and/or discrete) layers of materials. As an example, FIG. 2A depicts a web of intermediate pant product 140, such as that folded by machine 118, discussed below; and FIG. 2B depicts a finished, unfolded product 146. Generally, and as introduced above, each of the pant products is formed from a first waistband 150, a second waistband 160, and a core 170 that also collectively define a first (or inner) face (or surface) 142 and a second (or outer) face (or surface) 144. Typically, the inner face 142 is oriented towards the user when wearing the finished product 146 and the outer face 144 is orientated away from the user when wearing the finished product 146.

Referring to FIG. 2A, the intermediate pant product 140 is formed of one or more continuous webs of the layers that form the waistbands (or waistband materials) 150, 160 along a longitudinal dimension with discrete cores 170 generally extending in a lateral direction between a pair of waistbands 150, 160, e.g., such that a first end 172 of each core 170 overlaps and/or is secured with the first waistband 150, a second end 174 of each core 170 overlaps and/or is secured with the second waistband 160, and a center portion 176 of each core 170 extends in between. As shown, each of the first and/or second waistbands 150, 160 may be considered to have upper edges 152, 162 extending along the distal ends of the continuous web (and forming the upper edges of the finished product 146) and leg contours 154, 164 proximate to cores 170 (and that form the leg holes of the finished product 146). As described in greater detail below, the intermediate pant product 140 may be provided to the folding machine 118 (FIG. 1) in order to form a more symmetrical tri-fold structure in which the waistbands 150, 160 are folded onto each other and the core 170 is tucked up in between the waistbands 150, 160. Subsequently, the waistbands 150, 160 are bonded to form side seams 148 and separated at cut lines 149, as examples of additional processing steps, to eventually form the finished pant product, such as the pant product 146 of FIG. 2B. Additional views and discussion of the folding of the intermediate pant product 140 are provided below.

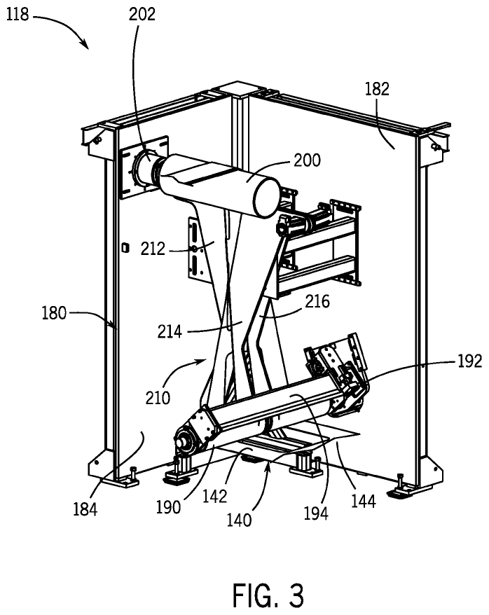

The views of FIGS. 3-8 depict various aspects of folding machine 118 that operate to make three folds in the intermediate pant product 140 to result in an “M” or “W” shape, as discussed in greater detail below.

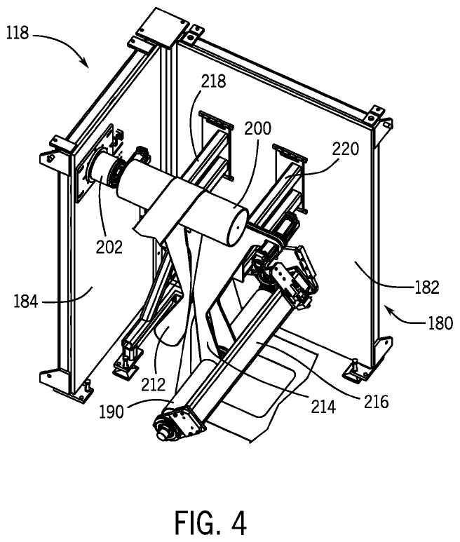

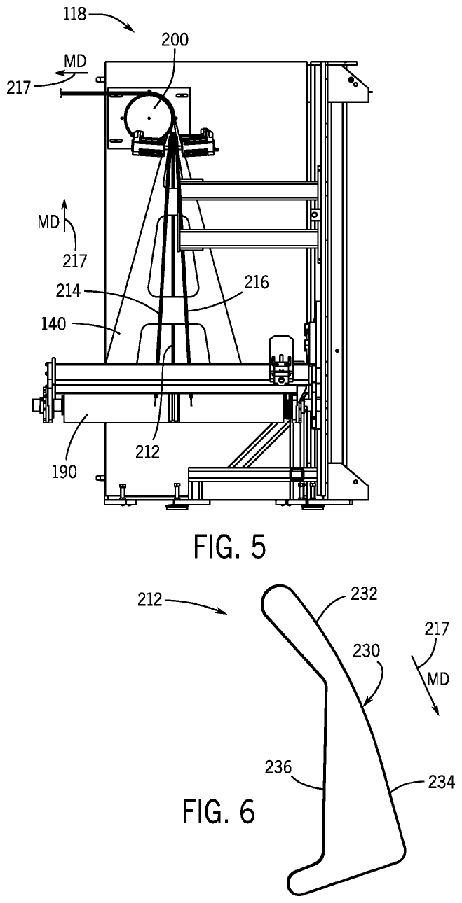

In the depicted example, FIGS. 3 and 4 may be considered side isometric views of the folding machine (or apparatus or unit) 118 that receives a continuous stream of waistbands and cores from section 120 (e.g., similar to intermediate pant product 140); and FIG. 5 is a side view of the folding machine 118 oriented along the direction of travel of the incoming intermediate pant product. Generally, the folding machine 118 may be considered to include a frame or base 180 that supports first and second rollers 190, 200 and a folding apparatus 210. In this example, the incoming intermediate pant product 140 is unfolded and “face-up” such that the inner face 142 is oriented to engage the first roller and the outer face 144 faces toward the ground; and the outgoing intermediate pant product 140 is folded along three folds such that the outer face 144 faces outward in each transverse direction. As described in greater detail below, the orientation and configuration of the intermediate pant product 140 into and out of the folding machine 118 may be modified as necessary or desired to result in the desired intermediate or finished product.

In one example, the frame 180 may form a first mounting structure 182 and a second mounting structure 184. In this example, the first mounting structure 182 is perpendicular to the second mounting structure 184 and is parallel to the longitudinal direction of the incoming (or unfolded) intermediate pant product 140; and the second mounting structure 184 is oriented generally parallel to the longitudinal direction of the outgoing (or folded) intermediate pant product 140.

The first roller 190 may be considered an inlet roller and is supported on and extends from the first mounting structure 182 via a first roller support 192 to receive and guide the incoming intermediate pant product 140 in an unfolded state to the folding apparatus 210. A first roller guide 194 may also be supported on the first mounting structure 182 to facilitate guidance of the intermediate pant product 140 to the folding apparatus 210. The second roller 200 may be considered an outlet roller and is supported on and extends from the second mounting structure 184 via a second roller support 202 to receive the intermediate pant product 140 in the folded state from the folding apparatus 210 and to guide the outgoing intermediate pant product 140 to a subsequent or downstream section (e.g., section 124).

In some examples, one or more of the rollers 190, 200 may be powered to rotate in order to facilitate or drive movement of the intermediate pant product 140, while in other examples, the rollers 190, 200 may be unpowered (e.g., “fly wheels”) to be driven by the movement of the intermediate pant product 140. In still other examples, the rollers 190, 200 may rotationally stationary and merely provide smooth surfaces that guide the intermediate pant product 140 into the desired positions and/or orientations.

The folding apparatus 210 may be formed by three folding boards 212, 214, 216 mounted on the first mounting structure 182 via supports 218, 220. As best shown in the side view of FIG. 5 in which the machine direction (MD) 217 is indicated, the inlet roller is arranged perpendicularly to the outlet roller 200 such that the incoming intermediate pant product 140 is redirected in both a vertical direction and a perpendicular horizontal direction to result in the position of the outgoing intermediate pant product 140. However, other arrangements and/or configurations may be provided, particularly depending on the context of preceding and subsequent stations (e.g., stations 120 and 124, respectively, of FIG. 1). For example, in a further configuration, the intermediate pant product 140 may enter and exit the folding apparatus 210 and/or the folding machine 118 along a relatively common straight line (e.g., rather than making an orthogonal turn). In this example, the boards 212, 214, 216 are stationary, although as discussed in greater detail below, additional mechanisms may be provided to facilitate folding and transfer.

An example folding board (e.g., board 212) is depicted in FIG. 6, isolated from the folding machine 118 for clarity. The center board 212 depicted in FIG. 6 is also representative of boards 214, 216. In one example, the boards 212, 214, 216 are shaped and sized identically, albeit with different orientations. In other examples, the boards 212, 214, 216 may be different from one another.

As shown, the board 212 is relatively flat and relatively thin with opposing planar surfaces so as to define a board engagement edge 230 and an opposite distal edge 236. The board engagement edge 230 may be considered to include a guiding edge portion (or nose) 232 and a folding edge portion 234, one slightly angled relative to the other. Upon installation in the machine 118, the board 212 is oriented such that the board engagement edge 230 is generally oriented along the longitudinal orientation. Generally, and as discussed in greater detail below, the guiding edge portion 232 may receive the intermediate pant product 140 and transition the intermediate pant product 140 to the folding edge portion 234 such that adjacent portions of the material are pressed onto and around the edge portion 234 to form the respective fold. The other boards 214, 216 operate in a similar manner, and collectively, the boards 212, 214, 216 operate to form three folds, as discussed in greater detail below.

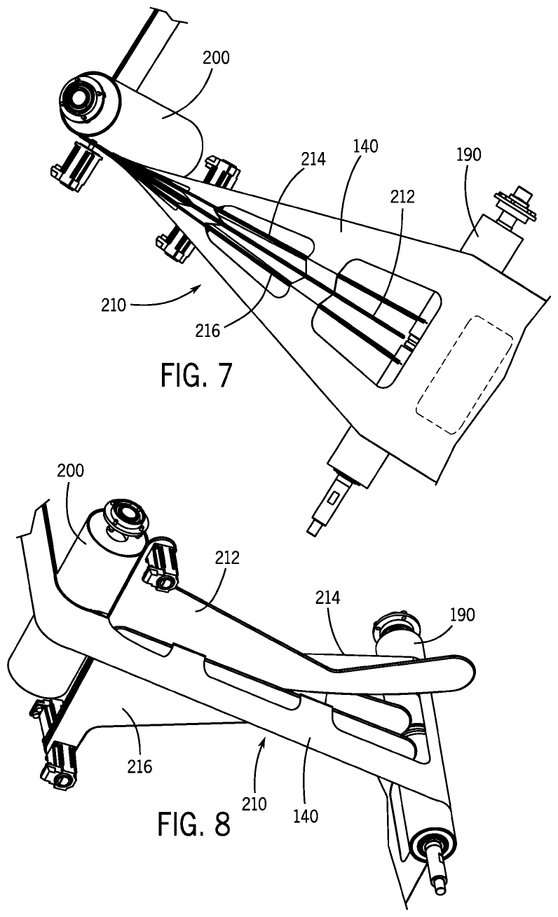

The views of FIGS. 7 and 8 provide additional perspectives of the rollers 190, 200 and the boards 212, 214, 216. As shown, the boards 212, 214, 216 are arranged such that the center board 212 is arranged in between the side boards 214, 216. Additionally, in the depicted orientation of the incoming intermediate pant product 140, the individual boards 212, 214, 216 are arranged and positioned such that the folding edge of the center board 212 engages the outer face 144 of the intermediate pant product 140 and the folding edges of the side boards 214, 216 engage the inner face 142 of the intermediate pant product 140. The side boards 214, 216 are arranged so as to converge towards the center board 212 along the direction of product movement, e.g., a wider spacing at forward or upstream ends and a narrower spacing at rear or downstream ends. The converging spacing functions to complete the folds of the intermediate pant product 140 along the boards 212, 214, 216.

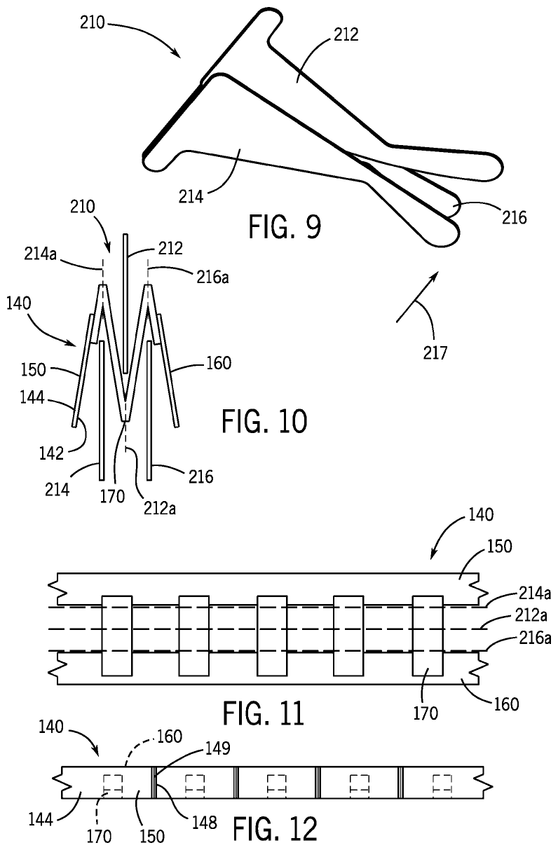

A more detailed description of the folding process of the folding apparatus 210 is provided by the views of FIGS. 9-12. In particular, FIG. 9 is an isolated view of the three boards 212, 214, 216 of the folding apparatus 210. Additionally, FIG. 10 is a cross-sectional view of the folding apparatus 210 and intermediate pant product 140 in a lateral-transverse plane as the product 140 is being folded by the boards 212, 214, 216. Reference is also made to FIG. 11, which is a simplified schematic view of the intermediate pant product 140. In particular, FIG. 11 depicts the inner face 142 of the intermediate pant product 140, including the first and second waistbands 150, 160 and the cores 170. The views of FIGS. 10 and 11 additionally include dashed lines 212a, 214a, 216a that schematically represent the positions of the boards 212, 214, 216 as the boards 212, 214, 216 engage and form folds in the intermediate pant product 140.

As noted, the boards 212, 214, 216 may have similar shapes. However, in this example, the center board 212 is arranged such that an engagement edge is facing generally in a first direction (e.g., generally upward) and the side boards 214, 216 are arranged such that respective engagement edges are facing generally in a second direction (e.g., generally downward). This arrangement enables the intermediate pant product 140 to pass in between and engage the engagement edges of the center board and the side boards 214, 216 to enable three folds along the longitudinal orientation.

In some examples, the center board 212 is positioned slightly forward of the side boards 214, 216 relative to the machine direction (MD) 217 so as to initially engage the intermediate pant product prior to the side boards 214, 216, although in other examples, the boards 212, 214, 216 may have forward ends that are aligned with respect to the upstream longitudinal position. In any event, the boards 212, 214, 216 are arranged such that at least a portion of the folding occurs simultaneously with respect to all of the three boards 212, 214, 216, e.g., along at least a common portion of the overall longitudinal path.

As best shown by reference to FIGS. 10 and 11, the center board 212 engages the outer face 144 of the intermediate pant product 140 at line 212a, roughly in the lateral middle, of the core 170 to form a center fold; and the side boards 214, 216 engage the inner face 142 of the intermediate pant product 140 at lines 214a, 216a, approximately in the position at which the core 170 overlaps each respective waistband 150, 160, to form first and second side folds.

Accordingly, the arrangement of the boards 212, 214, 216 results in the core 170 being at least partially “tucked” up in between the inward (user-contacting) faces of waistband materials 150, 160, thereby providing an “M-shape” in cross-section (or a “W-shape”, depending on perspective), as best shown in FIG. 10. In some examples, the entire mass of the core 170 may be arranged in between the waistbands 150, 160, although the exact positions of the side folds may vary based on product size and design.

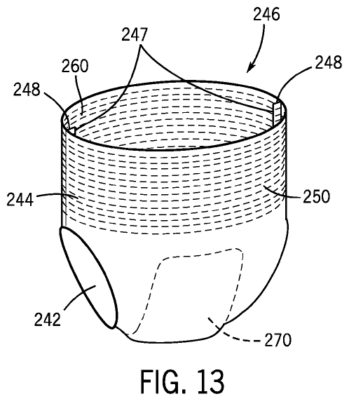

Reference is additionally made to FIG. 12, which depicts a simplified schematic side view of the resulting folded intermediate pant product 140, e.g., as it is transferred to and around the second roller 200. As shown, the folded intermediate pant product 140 is configured such that outer faces of the waistbands 150, 160 are outwardly facing in the transverse orientation, and the cores 170 are at least partially tucked in between. The view of FIG. 12 additionally depicts a schematic dashed representation 148 of the seams (subsequently formed by ultrasonic or adhesive bonding) and cuts 149 (also subsequently formed) that result in individual finished pant products 146 (FIG. 2B). Because the seams 148 are formed with the outer face 144 of the intermediate pant product 140 facing outward, the fin-portion 147 of the side seam 148 is located on the outside of the finished product 146, as shown in FIG. 2B. The folded, bonded, and separated pant products 140 may be folded again, e.g., along lateral lines, in a manner suitable for packaging. Generally, any further processing may be provided for the folded intermediate pant product 140 prior to being a finished product that is packaged and distributed. For example, in some configurations, the folded intermediate pant product may be partially unfolded to untuck the core 170; however, in other examples, the folded intermediate pant product 140 may be maintained in the “W-shape” or “M-shape” configuration, which may provide a more symmetrical distribution of mass so as to facilitate downstream handing, as well as a more robust continuous web of cores and waistband material, thereby enabling faster manufacturing speeds and efficiencies. The folding apparatus 210 may have various arrangements to provide the tri-fold configuration of the resulting folded intermediate pant product 140. As a further example, reference is made to FIGS. 13-19 in which an alternate folding apparatus 310 may be used to manufacture a further folded intermediate pant product 240, thereby providing a finished unfolded pant product 246.

Initially, the finished, unfolded product 246 is depicted in the isometric view of FIG. 13. Generally, and as introduced above, each of the pant products is formed from a first waistband 250, a second waistband 260, and a core 270 that also collectively define a first (or inner) face (or surface) 242 and a second (or outer) face (or surface) 244. Typically, the inner face 242 is oriented towards the user when wearing the finished product 246 and the outer face 244 is orientated away from the user when wearing the finished product 246. The waistbands 250, 260 are joined at seams 248, which in this example, are formed with the inner (user-contacting) face 242 of the intermediate pant product 240 facing outward, and the fin-portion 247 of the side seam 248 is located in the inside (user-facing surface) of the finished product 246.

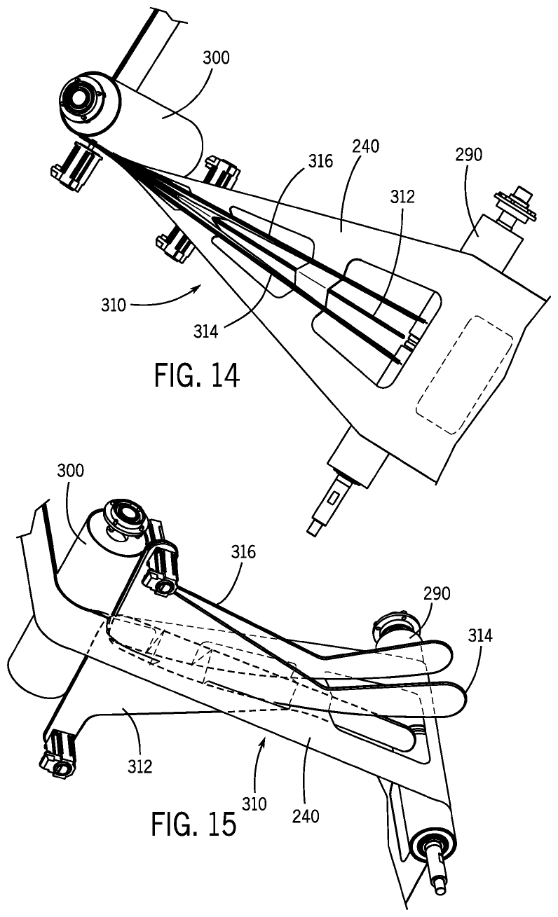

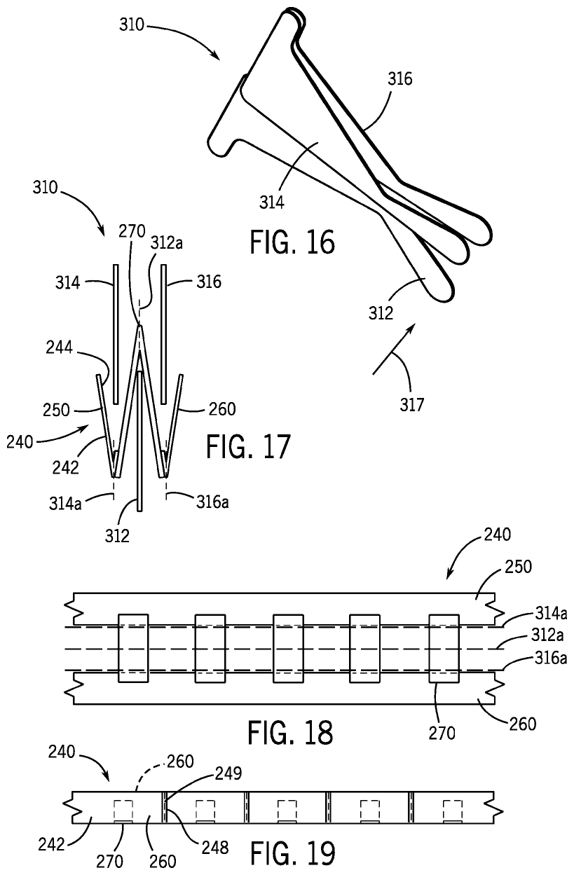

Further, FIGS. 14 and 15 provide isometric views of the alternate folding apparatus 310 arranged relative to rollers 290, 300 to fold a corresponding intermediate pant product 310; and FIG. 16 is an isometric view of the alternate folding apparatus 310 isolated from the rollers 290, 300, intermediate pant product 249, and other portions of a larger folding machine. Unless otherwise noted, the intermediate pant product 240 and a folding machine that incorporates the folding apparatus 310 of FIGS. 14-16 may be identical to (or different than) the folding machine 118 described above.

As above, the folding apparatus 310 may include a center board 312 and side boards 314, 316; and boards 312, 314, 316 may have similar shapes. However, relative to the example discussed above, the side boards 314, 316 in this example are arranged such that the engagement edges are facing generally in a first direction (e.g., generally upward) and the center board 312 is arranged such that engagement edge is facing generally in a second direction (e.g., generally downward). This arrangement enables the intermediate pant product 240 to pass in between and engage the engagement edges of the center board 312 and the side boards 314, 316 to enable three folds along the longitudinal orientation.

In this example, as above, the side boards 314, 316 are positioned slightly forward (upstream) of the side boards 314, 316 in the machine direction 217 so as to initially engage the intermediate pant product prior to the center board 312, although in other examples, the boards 312, 314, 316 may have forward ends that are aligned with respect to the upstream longitudinal position.

In this arrangement, and as better depicted in the cross-sectional view of FIG. 17 in which the intermediate pant product 240 is being folded by folding apparatus 310 and the simplified schematic view of FIG. 18 in which the engagement lines 312a, 314a, 316a of the corresponding boards 312, 314, 316 are shown, the center board 312 engages an inner face 242 of the intermediate pant product 240 at line 312a, approximately in the lateral middle at a core 270, to form a center fold; and the side boards 314, 316 engage the outer face 244 of the intermediate pant product 240 at lines 314a, 316a, approximately in the position at which the core 270 overlaps each respective waistband 250, 260, to form first and second side folds.

As above, this results in the core 270 being at least partially “tucked” up in between the waistband materials 250, 260, thereby providing a “W-shape” in cross-section (or an “M-shape”, depending on perspective), as best shown in FIG. 17. However, in this example, the folded intermediate pant product 240 has the inner face facing transversely outward. In some examples, the entire mass of the core 270 may be arranged in between the waistbands 250, 260, although the exact positions of the side folds may vary based on product specifications.

Reference is additionally made to FIG. 19, which depicts a simplified schematic side view of the resulting folded intermediate pant product 240, e.g., as it is transferred to and around the second roller 200. As shown, the folded intermediate pant product 240 is configured such that inner faces of the waistband 250, 260 are outwardly facing in the transverse orientation, and the cores 270 are tucked in between. In some embodiments, a portion of the user-facing surfaces of the cores 270 are also outwardly facing in the transverse orientation, as shown in FIG. 19. One skilled in the art will recognize that the length of the portion of the cores 270 facing outwardly will vary based on product size and design. The view of FIG. 19 additionally depicts a schematic dashed representation of the seams (subsequently formed by ultrasonic bonding) and cuts 249 (also subsequently formed) that result in individual folded products. The folded, bonded, and separated products 240 may be folded again, e.g., along lateral lines, in a manner suitable for packaging. Because the seams 248 are formed with the inner (user-contacting) face 242 of the intermediate pant product 240 facing outward, the fin-portion of the side seam 248 is located in the inside (user-facing surface) of the finished product 246, as shown in FIG. 13.

Generally, any further processing may be provided for the intermediate pant product 240 prior to being a finished product that is packaged and distributed. For example, in some configurations, the folded intermediate pant product 240 may be partially unfolded and/or refolded such that the outer face 244 faces outward in the transverse directions. In other words, the folded intermediate pant product 240 may be refolded from “inside-out” to “right-side out,” which is more typical of such finished products; however, in other examples, the folded intermediate pant product 240 may be maintained in the “inside-out” orientation, e.g., which may facilitate the user placement or donning of the finished product.

In any event, during the further processing of the folded intermediate pant product 240, the seams 248 are generally formed on the inner face 242 of the product since the bonding of the folded intermediate pant product 240 occurs to the inner face 242 that is facing transversely outward in this example. The resulting interior seams 248 of the finished pant product may provide a more desirable, garment-like consumer product.

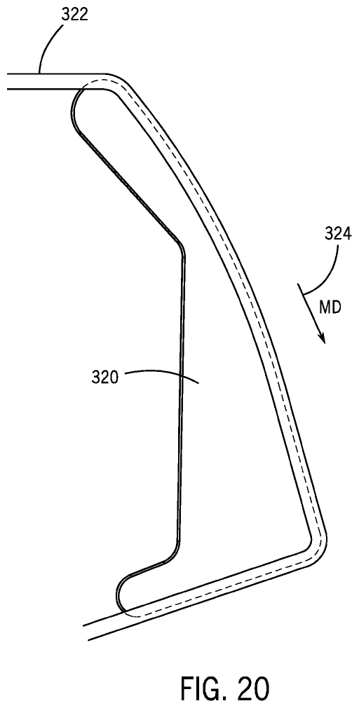

Further modifications and/or alternate configurations of the folding machine 118 may be provided. For example, bands or strips may be provided to cooperate with the boards to facilitate smooth passage of the intermediate product along the boards during folding. An example is provided by the view of FIG. 20, which is a sample folding board (e.g., corresponding to boards 212, 214, 216, 312, 314, 316) that may be incorporated into the folding machine 118 discussed above. As shown, a rotating band may be provided to travel along the perimeter at the engagement edge of the board in the direction of material movement corresponding to machine direction (MD) 324. Such a band 322 may be driven by a motor mounted to the frame (e.g., frame 180 of FIG. 1). The rotating band 322 may be applied to any one or more of the center or side boards to facilitate smooth transport of the material. In particular, the rotating band 322 may reduce friction between a center board and the core of the intermediate product, which may reduce or eliminate the possibility of the board tearing the core.

Accordingly, the example embodiments discussed herein provide folding machines, systems, and methods that may result in benefits with respect to process and packaging. In particular, the folded intermediate pant product resulting from the boards of the folding apparatus may provide a more symmetrical distribution of mass so as to facilitate downstream handing, as well as a more robust continuous web of cores and waistband material, thereby enabling faster manufacturing speeds and efficiencies. In some instances, by providing the three folds of the intermediate pant product approximately simultaneously with the cooperating boards, the overall footprint of the folding machine may be reduced. Moreover, examples described herein may facilitate or enable the formation of a finished pant product with inner side seams, thereby providing a more desirable consumer product.

Also, the following examples are provided, which are numbered for easier reference.
1. A pant folding machine configured to fold an intermediate pant product having a first face and a second face and formed by a continuous web of first waistband material, a continuous web of second waistband material, and a plurality of cores, each respective extending between the continuous web of first waistband material and the continuous web of second waistband material, the pant folding machine comprising: a frame; an inlet roller mounted on the frame and configured to receive the intermediate pant product in an unfolded state; a folding apparatus arranged downstream of the inlet roller, mounted on the frame, and configured to receive the intermediate pant product from the inlet roller in the unfolded state, the folding apparatus comprising a center board, a first side board on a first side of the center board, and a second side board on a second side of the center board, wherein the center board, the first side board, and the second side board are arranged such that the center board engages the first face of the intermediate pant product and each of the first and second side boards engages the second face of the intermediate pant product and, as the intermediate pant product is drawn along the center board and the first and second side boards, the intermediate pant product is folded by each of the center board and the first and second side boards to transition the intermediate pant product from the unfolded state to a folded state; and an outlet roller mounted on the frame and configured to receive the intermediate pant product in the folded state from the folding apparatus.
2. The pant folding machine of example 1, wherein the inlet roller and the folding apparatus are arranged such that the center board engages the cores of the intermediate pant product.
3. The pant folding machine of example 2, wherein the inlet roller and the folding apparatus are arranged such that the center board engages the cores of the intermediate pant product at approximately a lateral center.
4. The pant folding machine of example 3, wherein the inlet roller and the folding apparatus are arranged such that the first side board engages the intermediate pant product proximate to a first respective interface of the core and the first waistband material and such that the second side board engages the intermediate pant product proximate to a second respective interface of the core and the second waistband material.
5. The pant folding machine of example 1, wherein the folding apparatus is configured such that the intermediate pant product is folded into an M-shape or a W-shape.
6. The pant folding machine of example 1, wherein the folding apparatus is configured such that the cores of the intermediate pant product are tucked up in between the first waistband material and the second waistband material.
7. The pant folding machine of example 1, wherein the inlet roller is arranged perpendicularly relative to the outlet roller.
8. The pant folding machine of example 1, wherein the inlet roller and folding apparatus are arranged such that the center board engages the first face as an outer face of the intermediate pant product and the side boards engage the second face as an inner face of the intermediate pant product.
9. The pant folding machine of example 1, wherein the inlet roller and folding apparatus are arranged such that the center board engages the first face as an inner face of the intermediate pant product and the side boards engage the second face as an outer face of the intermediate pant product.
10. The pant folding machine of example 1, wherein the center board, the first side board, and the second side board are arranged so as to each engage the intermediate pant product along a common longitudinal path.
11. The pant folding machine of example 1, wherein the center board, the first side board, and the second side board each have a first end oriented proximate to the inlet roller and a second end oriented proximate to the outlet roller, and wherein the first ends of the center board, the first side board, and the second side board have a first lateral spacing and the second ends of the center board, the first side board, and the second side board have a second lateral spacing, the first lateral spacing being greater than the second lateral spacing.
12. The pant folding machine of example 1, wherein the center board has relatively planar side faces that meet to define an edge to engage the intermediate pant product.
13. The pant folding machine of example 1, wherein the center board, the first side board, and the second side board have generally identical shapes.
14. The pant folding machine of example 1, wherein the center board, the first side board, and the second side board are stationary.
15. The pant folding machine of example 1, further comprising a transport band extending along at least a perimeter portion of the center board, the transport band configured to be driven in a direction of movement of the intermediate pant product.
16. A method for folding an intermediate pant product defining a first face and a second face and having a first waistband, a second waistband, and a core, the method comprising: receiving the intermediate pant product in an unfolded state; directing the intermediate pant product along a folding apparatus with a center board, a first side board on a first side of the center board, and a second side board on a second side of the center board such that that the center board engages the first face of the intermediate pant product to form a center fold and each of the first and second side boards engages the second face of the intermediate pant product to respectively form first and second side folds; and directing the intermediate pant product away from the folding apparatus in a folded state.
17. A method of forming a diaper-type pant product, the method comprising: forming and providing a continuous web of first waistband material; forming and providing a continuous web of second waistband material; positioning a series of cores at least partially on the continuous web of first waistband material and at least partially on the continuous web of second waistband material to form an intermediate pant product with a first face and a second face; folding the intermediate pant product with at least two folding boards to result in a folded intermediate pant product; bonding the continuous web of first waistband material and the continuous web of second waistband material of the folded intermediate pant product to create a series of side seams; and cutting the bonded continuous webs proximate the side seams to form a series of discrete pant products.
18. The method of example 17, wherein the folding step includes creating a center fold with a center board of the at least two folding boards at the cores on the first face of the intermediate pant product; creating a first side fold with a first side board of the at least two folding boards proximate to an interface between the cores and the continuous web of first waistband material on the second face of the intermediate pant product; and creating a second side fold with a second side board of the at least two folding boards proximate to an interface between the cores and the continuous web of second waistband material on the second face of the intermediate pant product.
19. The method of example 18, wherein the first face of the intermediate pant product is an outer face and the second face of the intermediate pant product is an inner face such that the bonding and cutting steps operate to form the discrete pant products with the side seams as outer side seams.
20. The method of example 18, wherein the first face of the intermediate pant product is an inner face and the second face of the intermediate pant product is an outer face such that the bonding and cutting steps operate to form the discrete pant products with the side seams as inner side seams.
21. A folded diaper-type pant product, comprising: a first waistband; a second waistband bonded to the first waistband with inner seams; and a core, extending between and at least partially overlapping the first waistband and the second waistband, the core having a first fold in a lateral center such that the core is tucked up in between the first waistband and the second waistband to additionally form a first side fold proximate to an interface of the core and the first waistband and a second side fold proximate to an interface of the core and the second waistband.

The detailed description herein describes exemplary embodiments and is not intended to be limited to the expressly disclosed combination(s). Therefore, unless otherwise noted, features disclosed herein may be combined together to form additional combinations that were not otherwise shown for purposes of brevity. As such, references to a feature or aspect are intended to describe a feature or aspect of an example, not to imply that every embodiment thereof must have the described feature or aspect. Furthermore, it should be noted that the description illustrates a number of features. While certain features have been combined together to illustrate potential system designs, those features may also be used in other combinations not expressly disclosed. Thus, the depicted combinations are not intended to be limiting, unless otherwise noted.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, unless otherwise limited or modified, lists with elements that are separated by conjunctive terms (e.g., “and”) and that are also preceded by the phrase “one or more of” or “at least one of” indicate configurations or arrangements that potentially include individual elements of the list, or any combination thereof. For example, “at least one of A, B, and C” or “one or more of A, B, and C” indicates the possibilities of only A, only B, only C, or any combination of two or more of A, B, and C (e.g., A and B; B and C; A and C; or A, B, and C).

The description of the present disclosure has been presented for purposes of illustration and description, but it is not intended to be exhaustive or limited to the disclosure in the form disclosed. Many modifications and variations will be apparent to those of ordinary skill in the art without departing from the scope and spirit of the disclosure. Explicitly referenced embodiments herein were chosen and described in order to best explain the principles of the disclosure and their practical application, and to enable others of ordinary skill in the art to understand the disclosure and recognize many alternatives, modifications, and variations on the described example(s). Accordingly, various embodiments and implementations other than those explicitly described are within the scope of the claims.

1. A pant folding machine configured to fold an intermediate pant product having a first face and a second face and formed by a continuous web of first waistband material, a continuous web of second waistband material, and a plurality of cores, each respective extending between the continuous web of first waistband material and the continuous web of second waistband material, the pant folding machine comprising:
a frame;
an inlet roller mounted on the frame and configured to receive the intermediate pant product in an unfolded state;
a folding apparatus arranged downstream of the inlet roller, mounted on the frame, and configured to receive the intermediate pant product from the inlet roller in the unfolded state, the folding apparatus comprising
a center board,
a first side board on a first side of the center board, and
a second side board on a second side of the center board,
wherein the center board, the first side board, and the second side board are arranged such that the center board engages the first face of the intermediate pant product and each of the first and second side boards engages the second face of the intermediate pant product and, as the intermediate pant product is drawn along the center board and the first and second side boards, the intermediate pant product is folded by each of the center board and the first and second side boards to transition the intermediate pant product from the unfolded state to a folded state; and
an outlet roller mounted on the frame and configured to receive the intermediate pant product in the folded state from the folding apparatus.

2. The pant folding machine of claim 1, wherein the inlet roller and the folding apparatus are arranged such that the center board engages the plurality of cores of the intermediate pant product.

3. The pant folding machine of claim 2, wherein the inlet roller and the folding apparatus are arranged such that the center board engages the plurality of cores of the intermediate pant product at approximately a lateral center.

4. The pant folding machine of claim 3, wherein the inlet roller and the folding apparatus are arranged such that the first side board engages the intermediate pant product proximate to a first respective interface of the plurality of cores and the first waistband material and such that the second side board engages the intermediate pant product proximate to a second respective interface of the plurality of cores and the second waistband material.

5. The pant folding machine of claim 1, wherein the folding apparatus is configured such that the intermediate pant product is folded into an M-shape or a W-shape.

6. The pant folding machine of claim 1, wherein the folding apparatus is configured such that the cores of the intermediate pant product are tucked up in between the first waistband material and the second waistband material.

7. The pant folding machine of claim 1, wherein the inlet roller is arranged perpendicularly relative to the outlet roller.

8. The pant folding machine of claim 1, wherein the inlet roller and folding apparatus are arranged such that the center board engages the first face as an outer face of the intermediate pant product and the first and second side boards engage the second face as an inner face of the intermediate pant product.

9. The pant folding machine of claim 1, wherein the inlet roller and folding apparatus are arranged such that the center board engages the first face as an inner face of the intermediate pant product and the first and second side boards engage the second face as an outer face of the intermediate pant product.

10. The pant folding machine of claim 1, wherein the center board, the first side board, and the second side board are arranged so as to each engage the intermediate pant product along a common longitudinal path.

11. The pant folding machine of claim 1, wherein the center board, the first side board, and the second side board each have a first end oriented proximate to the inlet roller and a second end oriented proximate to the outlet roller, and wherein the first ends of the center board, the first side board, and the second side board have a first lateral spacing and the second ends of the center board, the first side board, and the second side board have a second lateral spacing, the first lateral spacing being greater than the second lateral spacing.

12. The pant folding machine of claim 1, wherein the center board has relatively planar side faces that meet to define an edge to engage the intermediate pant product.

13. The pant folding machine of claim 1, wherein the center board, the first side board, and the second side board have generally identical shapes.

14. The pant folding machine of claim 1, wherein the center board, the first side board, and the second side board are stationary.

15. The pant folding machine of claim 1, further comprising a transport band extending along at least a perimeter portion of the center board, the transport band configured to be driven in a direction of movement of the intermediate pant product.

16. A method for folding an intermediate pant product defining a first face and a second face and having a first waistband, a second waistband, and a core, the method comprising:
receiving the intermediate pant product in an unfolded state;
directing the intermediate pant product along a folding apparatus with a center board, a first side board on a first side of the center board, and a second side board on a second side of the center board such that that the center board engages the first face of the intermediate pant product to form a center fold and each of the first and second side boards engages the second face of the intermediate pant product to respectively form first and second side folds; and
directing the intermediate pant product away from the folding apparatus in a folded state.

17. The method of claim 16, further comprising:
forming and providing a continuous web of first waistband material;
forming and providing a continuous web of second waistband material;
positioning a series of cores at least partially on the continuous web of first waistband material and at least partially on the continuous web of second waistband material to form the intermediate pant product with the first face and the second face;
bonding the continuous web of first waistband material and the continuous web of second waistband material of the folded intermediate pant product to create a series of side seams; and
cutting the bonded continuous webs proximate the side seams to form a series of discrete pant products.

18. The method of claim 17, wherein the step of directing the intermediate pant product along the folding apparatus creates a first side fold proximate to an interface between the cores and the continuous web and a second side fold proximate to an interface between the series of cores and the continuous web of second waistband material on the second face of the intermediate pant product.

19. The method of claim 18, wherein the first face of the intermediate pant product is an outer face and the second face of the intermediate pant product is an inner face such that the bonding and cutting steps operate to form the discrete pant products with the side seams as outer side seams.

20. The method of claim 18, wherein the first face of the intermediate pant product is an inner face and the second face of the intermediate pant product is an outer face such that the bonding and cutting steps operate to form the discrete pant products with the side seams as inner side seams.

21. A folded diaper-type pant product, comprising:
a first waistband;
a second waistband bonded to the first waistband with inner seams; and
a core, extending between and at least partially overlapping the first waistband and the second waistband, the core having a first fold in a lateral center such that the core is tucked up in between the first waistband and the second waistband to additionally form a first side fold proximate to an interface of the core and the first waistband and a second side fold proximate to an interface of the core and the second waistband.
